(19) United States
(12) Patent Application Publication
(10) Pub. No.: US 2024/0252032 A1
(43) Pub. Date: Aug. 1, 2024

(54) LAPAROSCOPIC SYSTEM AND SURGICAL ROBOTIC SYSTEM WITH LAPAROSCOPIC SYSTEM

(71) Applicant: Covidien LP, Mansfield, MA (US)

(72) Inventors: K V S Manoj Kumar Vadali, Hyderabad (IN); Sai Gautham Ravipati, Hyderabad (IN); Aadarsh Gupta, Bhopal (IN)

(21) Appl. No.: 18/407,570

(22) Filed: Jan. 9, 2024

Related U.S. Application Data
(60) Provisional application No. 63/441,218, filed on Jan. 26, 2023.

(51) Int. Cl. A61B 1/313; A61B 1/00; A61B 1/04; A61B 1/045; A61B 17/34; A61B 34/30

(52) U.S. Cl. CPC A61B 1/3132; A61B 1/00105; A61B 1/00154; A61B 1/042; A61B 1/045; A61B 17/3423; A61B 34/30; A61B 2034/2051; A61B 2034/302

(57) ABSTRACT

A laparoscope, a laparoscopic system, and a robotic system including a camera module configured to switch between a first mode of operation and a second mode of operation based on a sensor signal. The sensor signal may be generated by at least one of a magnetic sensor or a light sensor.

### CROSS-REFERENCE TO RELATED APPLICATIONS

This application claims the benefit of, and priority to, U.S. Provisional Patent Application Ser. No. 63/441,218 filed on Jan. 26, 2023. The entire contents of the foregoing application are incorporated by reference herein.

### BACKGROUND

Surgical robotic systems are used in a variety of surgical procedures, including minimally invasive medical procedures. Some surgical robotic systems include a surgeon console controlling a surgical robotic arm and a surgical instrument having an end effector (e.g., forceps or grasping instrument) coupled to and actuated by the robotic arm. In operation, the robotic arm is moved to a position over a patient and then guides the surgical instrument into a small incision via a surgical port or a natural orifice of a patient to position the end effector of the surgical instrument at a work site within the patient's body.

For any electro-mechanical system, the reliability and life expectancy of the system and its components depends on a variety of factors, such as the temperature at which it is operated. Power consumption of surgical robotic systems causes heat generation which, in the long-term, reduces the life expectancy of the system, and in the short-term, increases the risk of system-failure during operation. Additionally, the excessive usage of bandwidth during operation of a remotely operated surgical robotic system poses a boundary to tele-surgical implementations. Further, in regard to patient privacy, it is desirable to have a system wherein the laparoscope automatically begins recording only upon entry into the trocar port or body cavity, and automatically stops recording upon removal from the trocar port or body cavity.

### SUMMARY

The popularity of laparoscopic surgeries has increased tremendously owing to enhanced safety and blood loss prevention, as well the minimally invasive nature of the surgery. However, the recorded data of the surgeries performed each year accumulates and is being used to train the novice professionals for patient care. In order to protect the confidentiality of the patients as well as the staff in these recordings, several solutions process the recorded video to mask out potentially confidential information. However, these solutions require that the surgeon record the complete high-definition surgical video which consumes memory, processing power, and bandwidth. The average operating time for a laparoscopic surgery is about 77 minutes, but an appropriate camera shooting at 4K resolution allows no more than 90 minutes of continuous recording. Additionally, high video resolutions translate to large sized digital files, requiring significant time and bandwidth for processing, transmission, and storage. For example, a 30 minute surgery shot at 4K resolution requires 5 GB of storage space. In some implementations, all the data is sent to a hub over Wi-Fi, and this aggravates the challenge of storage requirements by posing an equivalent constraint of bandwidth requirements.

This disclosure provides an approach based on magnetic and photo-electric sensors incorporated into components of the laparoscopic system to switch between modes of operation. A signal is provided to determine whether the camera is inside an access port (or otherwise within the surgical site) and the signal is used to switch the camera between different modes of operation, where some modes of operation transmit and/or record only the relevant data, both protecting confidentiality of the patient and individuals in the surgical setting and reducing the storage and network constraints imposed on the system overall and its individual components. This approach is computationally cheaper in comparison to the case of image-based processing techniques and decreases chances of error.

According to one embodiment of the present disclosure, a laparoscopic system is disclosed. The laparoscopic system includes an access port and a laparoscope configured to be inserted through the access port to access a surgical site. The access port includes a magnet. The laparoscope includes a camera module configured to capture video of the surgical site and switchable between a first mode and a second mode, and a magnetic sensor operably coupled to the camera module. The magnetic sensor is configured to cause the camera module to switch from operating in the first mode to operating in the second mode when the magnetic sensor is positioned in proximity to the magnet of the access port.

In an aspect, a proximal portion of the access port may define a proximal opening configured to receive a distal portion of the laparoscope therethrough. Additionally, or alternatively, the magnet may be a ring magnet defining an opening aligned with the proximal opening of the access port.

In an aspect, the magnetic sensor may include at least one of a reed switch or a hall effect sensor.

In an aspect, the magnetic sensor may be operably coupled to a distal portion of the laparoscope and the magnet may be disposed at a proximal portion of the access port, such that the magnetic sensor is configured to cause the camera module to switch from operating in the first mode to operating in the second mode upon insertion of the distal portion of the laparoscope through the proximal portion of the access port.

In an aspect, the camera module may be configured to capture video when in the second mode and not capture video when in the first mode.

In an aspect, the camera module may be configured to transmit captured video when in the second mode and not transmit captured video when in the first mode.

In an aspect, the camera module may be configured to transmit video in a first resolution when in the second mode and transmit video in a second resolution when in the first mode, where the first resolution is higher than the second resolution.

According to another aspect of the present disclosure, a laparoscope is provided. The laparoscope includes a proximal portion, a distal portion including an opening, and an elongated body extending between the proximal portion and the distal portion. The laparoscope further includes an optical fiber and a camera module including a light sensor. The camera module is configured to capture video of a surgical site and is switchable between a first mode and a second mode. The optical fiber extends from the camera module, through the elongated body, and to the opening of the distal portion and is configured to transmit light passing through the opening to the light sensor. The light sensor switches the camera module between operating in the first mode and operating in the second mode based on an amount light to which the light sensor is exposed.

In an aspect, the light sensor may be a light dependent resistor.

In an aspect, the camera module may be configured to capture video when in the second mode and not capture video when in the first mode.

In an aspect, the camera module may be configured to transmit captured video when in the second mode and not transmit captured video when in the first mode.

In an aspect, the camera module may be configured to transmit video in a first resolution when in the second mode and transmit video in a second resolution when in the first mode, where the first resolution is higher than the second resolution.

According to yet another aspect of the present disclosure, a laparoscope is disclosed. The laparoscope includes a camera module and at least one of a magnetic sensor or a light sensor operably coupled to the camera module. The camera module is configured to capture video of the surgical site and is switchable between a first mode and a second mode. The magnetic sensor is configured to cause the camera module to switch from operating in the first mode to operating in the second mode when the magnetic sensor is positioned in proximity to a magnet of an access port. The laparoscope may also include an optical fiber operably coupled to the light sensor and the light sensor may be configured to cause the camera module to switch between operating in the first mode and operating in the second mode based on an amount light to which the light sensor is exposed. The camera module may be configured to capture video when in the second mode and not capture video when in the first mode; transmit captured video when in the second mode and not transmit captured video when in the first mode; transmit video in a first resolution when in the second mode and transmit video in a second resolution when in the first mode, the first resolution being higher than the second resolution; or stop a recording of the captured video when switched from the second mode of operation to the first mode of operation.

In an aspect, a distal portion of the laparoscope may be configured to extend through a ring magnet of an access port to expose the magnetic sensor to a magnetic field of the ring magnet.

In an aspect, the magnetic sensor may include at least one of a reed switch or a hall effect sensor.

In an aspect, the light sensor may be a light dependent resistor.

In an aspect, a distal portion of the laparoscope may include an opening and a distal end of the optical fiber may be disposed at the opening.

In an aspect, the camera module may operate in the first mode until it is switched to the second mode.

According to another aspect of the disclosure, a method for switching a mode of operation of a laparoscope is provided. The method includes operating the laparoscope in a first mode of operation, where the laparoscope records video at a first resolution; and in response to at least one of the laparoscope crossing a magnetic field generated by a ring magnet, upon passing through the ring magnet, or the laparoscope detecting a change in light exposure above a predetermined threshold, switching the laparoscope from operating in the first mode of operation to a second mode of operation, where the laparoscope records video at a second resolution higher than the first resolution.

### BRIEF DESCRIPTION OF THE DRAWINGS

Various embodiments of the present disclosure are described herein with reference to the drawings wherein:

FIG. 1 is a schematic illustration of a surgical robotic system including a control tower, a console, and one or more surgical robotic arms each disposed on a mobile cart according to an embodiment of the present disclosure;

FIG. 2 is a perspective view of a surgical robotic arm of the surgical robotic system of FIG. 1 according to an embodiment of the present disclosure;

FIG. 3 is a perspective view of a mobile cart having a setup arm with the surgical robotic arm of the surgical robotic system of FIG. 1 according to an embodiment of the present disclosure;

FIG. 4 is a schematic diagram of a computer architecture of the surgical robotic system of FIG. 1 according to an embodiment of the present disclosure;

FIG. 5 is a plan schematic view of the surgical robotic system of FIG. 1 positioned about a surgical table according to an embodiment of the present disclosure;

FIG. 6 is a perspective view of a laparoscopic system according to an embodiment of the present disclosure; and

FIG. 7 is a perspective view of a laparoscope according to an embodiment of the present disclosure.

### DETAILED DESCRIPTION

Embodiments are described in detail with reference to the drawings, in which like reference numerals designate identical or corresponding elements in each of the several views.

The present disclosure is directed to laparoscopic systems, laparoscopes, and robotic surgical systems, devices, methods, and computer-readable media that reduce energy consumption, heat generation, and/or bandwidth expenditure of one or more components of the laparoscopic systems, laparoscopes, or robotic surgical system when such resources are needed least. More particularly, the present disclosure relates to components of a system that are switchable between different modes of operation, thereby reducing heat generation, extending the life of the components of the robotic surgical system, enhancing the performance and communication between the components of the robotic surgical system, and protecting the privacy of the patient and clinicians. The systems and methods described herein provide various techniques for detecting whether a laparoscope is positioned within a surgical site and, based on the detection, causing one or more components of the system to change its mode of operation. Utilizing the technologies, techniques, and embodiments described herein, users are provided with a safer operating environment in which to perform robotic surgeries, patients are afforded a safer environment in which to receive surgical treatment via robotic surgical systems, communication speed and reliability between the components of the robotic surgical system is increased, the life expectancy of the components of the robotic surgical system is extended, and the privacy of the patient and clinicians is protected.

As will be described in detail below, the present disclosure is directed to a surgical robotic system, which includes a surgeon console, a control tower, and one or more mobile carts having a surgical robotic arm coupled to a setup arm. The surgeon console receives user input through one or more interface devices, which are processed by the control tower as movement commands for moving the surgical robotic arm and an instrument and/or camera coupled thereto. Thus, the surgeon console enables teleoperation of the surgical arms and attached instruments/camera. The surgical robotic arm includes a controller, which is configured to process the movement commands and to generate torque commands for activating one or more actuators of the robotic arm, which would, in turn, move the robotic arm in response to the movement command.

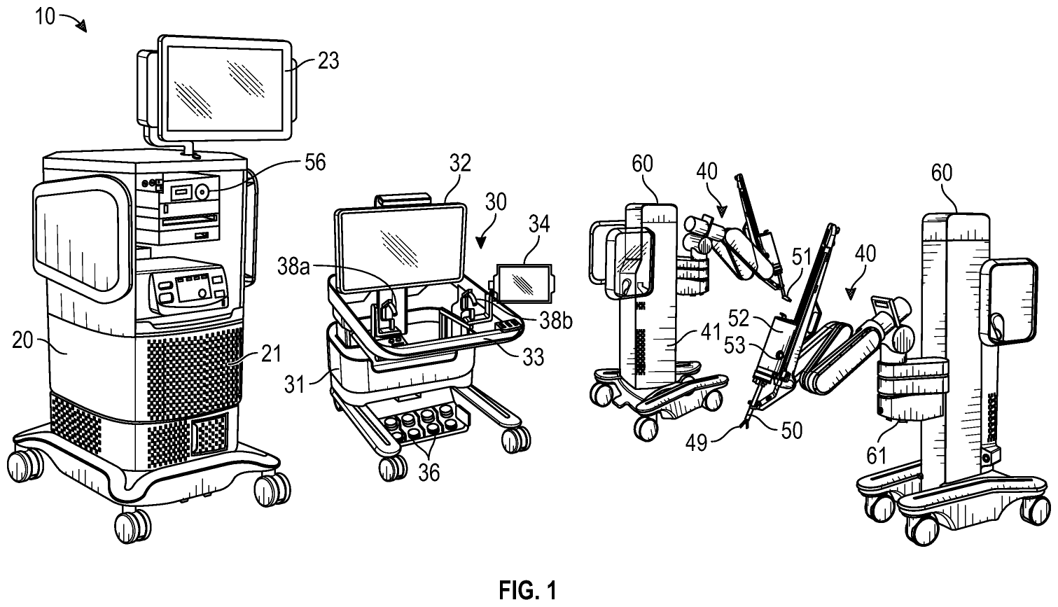

With reference to FIG. 1, a surgical robotic system 10 includes a control tower 20, which is connected to all of the components of the surgical robotic system 10 including a surgeon console 30 and one or more mobile carts 60. Each of the mobile carts 60 includes a robotic arm 40 having a surgical instrument 50 removably coupled thereto. The robotic arms 40 also couple to the mobile carts 60. The surgical robotic system 10 may include any number of mobile carts 60 and/or robotic arms 40.

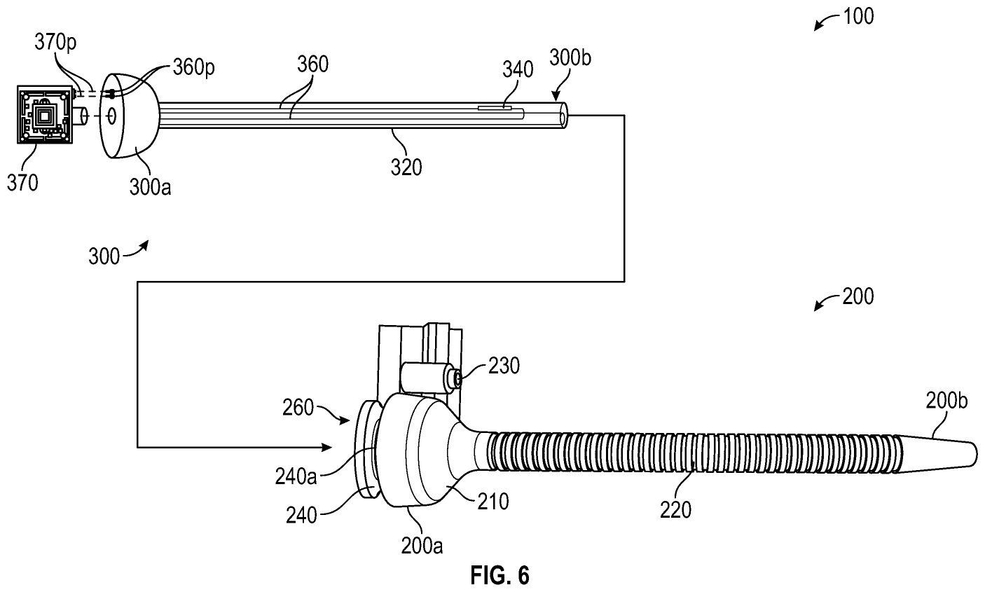

The surgical instrument 50 is configured for use during minimally invasive surgical procedures. In embodiments, the surgical instrument 50 may be configured for open surgical procedures. In further embodiments, the surgical instrument 50 may be an electrosurgical forceps configured to seal tissue by compressing tissue between jaw members and applying electrosurgical current thereto. In yet further embodiments, the surgical instrument 50 may be a surgical stapler including a pair of jaws configured to grasp and clamp tissue while deploying a plurality of tissue fasteners, e.g., staples, and cutting stapled tissue. In yet further embodiments, the surgical instrument 50 may be a surgical clip applier including a pair of jaws configured apply a surgical clip onto tissue. In yet further embodiments, the surgical instrument 50 may be an access port (e.g., access port 200 of FIG. 6) configured to enable access to the surgical site and receive other surgical instruments or imaging devices therethrough.

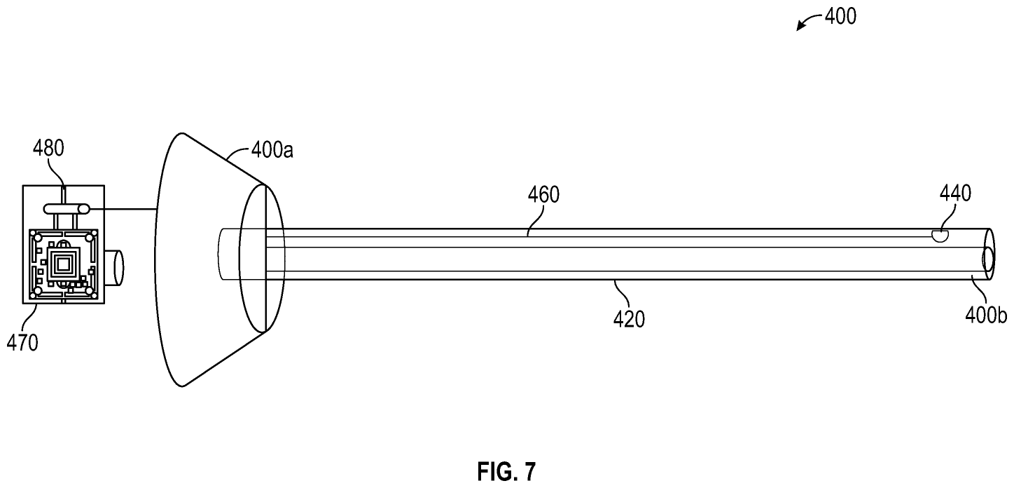

One of the robotic arms 40 may include a laparoscopic camera 51 (e.g., laparoscope 300 of FIG. 6 or laparoscope 400 of FIG. 7) configured to capture video of the surgical site. The laparoscopic camera 51 may be a stereoscopic endoscopic camera configured to capture two side-by-side (i.e., left and right) images of the surgical site to produce a video stream of the surgical scene. The laparoscopic camera 51 is coupled to a video processing device 56, which may be disposed within the control tower 20. The video processing device 56 may be any computing device configured to receive the video feed from the laparoscopic camera 51 and output the processed video stream.

The surgeon console 30 includes a first screen 32, which displays a video feed of the surgical site provided by laparoscopic camera 51 disposed on the robotic arm 40, and a second screen 34, which displays a user interface for controlling the surgical robotic system 10. The first screen 32 and second screen 34 may be touchscreens allowing for displaying various graphical user inputs.

The surgeon console 30 also includes a plurality of user interface devices, such as foot pedals 36 and a pair of hand controllers 38a and 38b which are used by a user to remotely control robotic arms 40. The surgeon console further includes an armrest 33 used to support clinician's arms while operating the hand controllers 38a and 38b.

The control tower 20 includes a screen 23, which may be a touchscreen, and outputs on the graphical user interfaces (GUIs). The control tower 20 also acts as an interface between the surgeon console 30 and one or more robotic arms 40. In particular, the control tower 20 is configured to control the robotic arms 40, such as to move the robotic arms 40 and the corresponding surgical instrument 50, based on a set of programmable instructions and/or input commands from the surgeon console 30, in such a way that robotic arms 40 and the surgical instrument 50 execute a desired movement sequence in response to input from the foot pedals 36 and the hand controllers 38a and 38b. The foot pedals 36 may be used to enable and lock the hand controllers 38a and 38b, repositioning camera movement and electrosurgical activation/deactivation. In particular, the foot pedals 36 may be used to perform a clutching action on the hand controllers 38a and 38b. Clutching is initiated by pressing one of the foot pedals 36, which disconnects (i.e., prevents movement inputs) the hand controllers 38a and/or 38b from the robotic arm 40 and corresponding surgical instrument 50 or laparoscopic camera 51 attached thereto. This allows the user to reposition the hand controllers 38a and 38b without moving the robotic arm(s) 40 and the surgical instrument 50 and/or laparoscopic camera 51. This is useful when reaching control boundaries of the surgical space.

Each of the control tower 20, the surgeon console 30, and the robotic arm 40 includes a respective computer 21, 31, 41. The computers 21, 31, 41 are interconnected to each other using any suitable communication network based on wired or wireless communication protocols. The term “network,” whether plural or singular, as used herein, denotes a data network, including, but not limited to, the Internet, Intranet, a wide area network, or a local area network, and without limitation as to the full scope of the definition of communication networks as encompassed by the present disclosure. Suitable protocols include, but are not limited to, transmission control protocol/internet protocol (TCP/IP), datagram protocol/internet protocol (UDP/IP), and/or datagram congestion control protocol (DC). Wireless communication may be achieved via one or more wireless configurations, e.g., radio frequency, optical, Wi-Fi, Bluetooth (an open wireless protocol for exchanging data over short distances, using short length radio waves, from fixed and mobile devices, creating personal area networks (PANs), ZigBee® (a specification for a suite of high level communication protocols using small, low-power digital radios based on the IEEE 122.15.4-1203 standard for wireless personal area networks (WPANs)).

The computers 21, 31, 41 may include any suitable processor (not shown) operably connected to a memory (not shown), which may include one or more of volatile, non-volatile, magnetic, optical, or electrical media, such as read-only memory (ROM), random access memory (RAM), electrically-erasable programmable ROM (EEPROM), non-volatile RAM (NVRAM), or flash memory. The processor may be any suitable processor (e.g., control circuit) adapted to perform the operations, calculations, and/or set of instructions described in the present disclosure including, but not limited to, a hardware processor, a field programmable gate array (FPGA), a digital signal processor (DSP), a central processing unit (CPU), a microprocessor, and combinations thereof. Those skilled in the art will appreciate that the processor may be substituted for by using any logic processor (e.g., control circuit) adapted to execute algorithms, calculations, and/or set of instructions described herein.

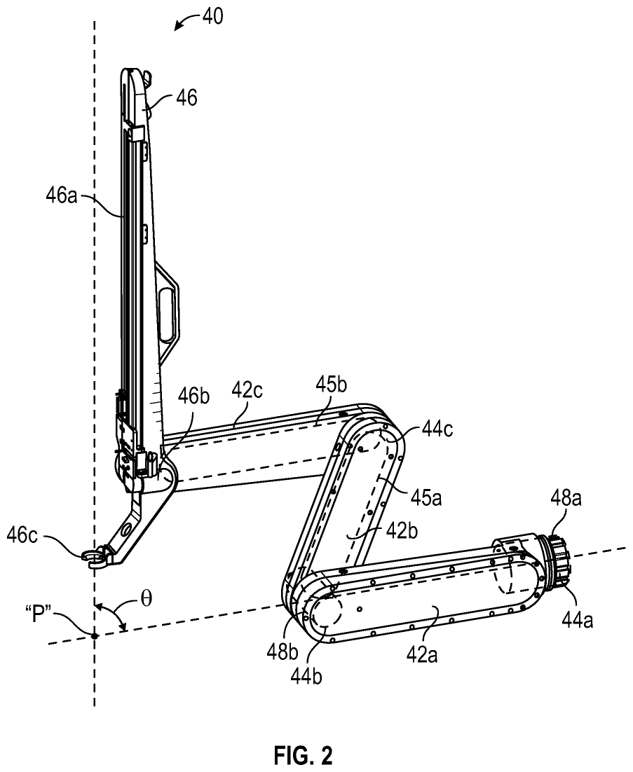

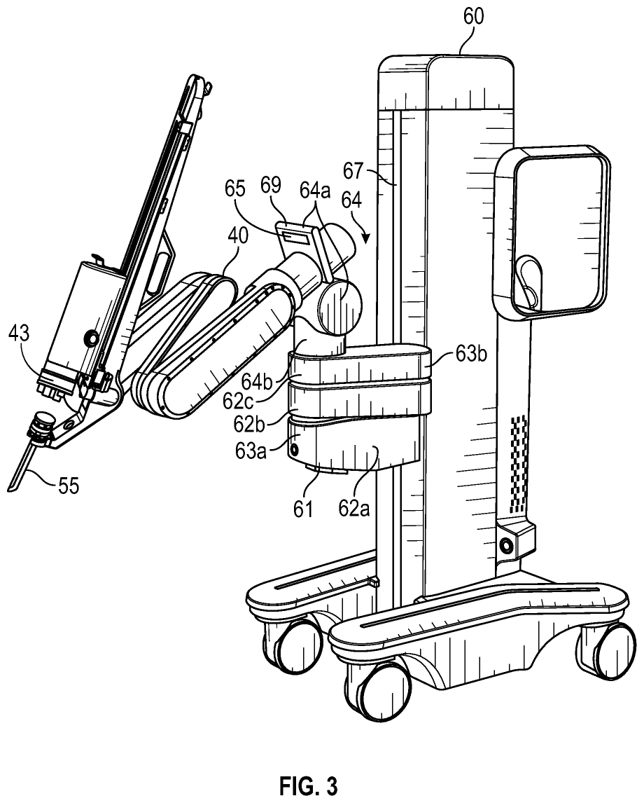

With reference to FIG. 2, each of the robotic arms 40 may include a plurality of links 42a, 42b, 42c, which are interconnected at joints 44a, 44b, 44c, respectively. Other configurations of links and joints may be utilized as known by those skilled in the art. The joint 44a is configured to secure the robotic arm 40 to the mobile cart 60 and defines a first longitudinal axis. With reference to FIG. 3, the mobile cart 60 includes a lift 67 and a setup arm 61, which provides a base for mounting of the robotic arm 40. The lift 67 allows for vertical movement of the setup arm 61. The mobile cart 60 also includes a screen 69 for displaying information pertaining to the robotic arm 40. In embodiments, the robotic arm 40 may include any type and/or number of joints.

The setup arm 61 includes a first link 62a, a second link 62b, and a third link 62c, which provide for lateral maneuverability of the robotic arm 40. The links 62a, 62b, 62c are interconnected at joints 63a and 63b, each of which may include an actuator (not shown) for rotating the links 62b and 62b relative to each other and the link 62c. In particular, the links 62a, 62b, 62c are movable in their corresponding lateral planes that are parallel to each other, thereby allowing for extension of the robotic arm 40 relative to the patient (e.g., surgical table). In embodiments, the robotic arm 40 may be coupled to the surgical table (not shown). The setup arm 61 includes controls 65 for adjusting movement of the links 62a, 62b, 62c as well as the lift 67. In embodiments, the setup arm 61 may include any type and/or number of joints.

The third link 62c may include a rotatable base 64 having two degrees of freedom. In particular, the rotatable base 64 includes a first actuator 64a and a second actuator 64b. The first actuator 64a is rotatable about a first stationary arm axis which is perpendicular to a plane defined by the third link 62c and the second actuator 64b is rotatable about a second stationary arm axis which is transverse to the first stationary arm axis. The first and second actuators 64a and 64b allow for full three-dimensional orientation of the robotic arm 40.

The actuator 48b of the joint 44b is coupled to the joint 44c via the belt 45a, and the joint 44c is in turn coupled to the joint 46b via the belt 45b. Joint 44c may include a transfer case coupling the belts 45a and 45b, such that the actuator 48b is configured to rotate each of the links 42b, 42c and a holder 46 relative to each other. More specifically, links 42b, 42c, and the holder 46 are passively coupled to the actuator 48b which enforces rotation about a pivot point “P” which lies at an intersection of the first axis defined by the link 42a and the second axis defined by the holder 46. In other words, the pivot point “P” is a remote center of motion (RCM) for the robotic arm 40. Thus, the actuator 48b controls the angle θ between the first and second axes allowing for orientation of the surgical instrument 50. Due to the interlinking of the links 42a, 42b, 42c, and the holder 46 via the belts 45a and 45b, the angles between the links 42a, 42b, 42c, and the holder 46 are also adjusted in order to achieve the desired angle θ. In embodiments, some or all of the joints 44a, 44b, 44c may include an actuator to obviate the need for mechanical linkages.

The joints 44a and 44b include an actuator 48a and 48b configured to drive the joints 44a, 44b, 44c relative to each other through a series of belts 45a and 45b or other mechanical linkages such as a drive rod, a cable, or a lever and the like. In particular, the actuator 48a is configured to rotate the robotic arm 40 about a longitudinal axis defined by the link 42a.

With reference to FIG. 2, the holder 46 defines a second longitudinal axis and configured to receive an instrument drive unit (IDU) 52 (FIG. 1). The IDU 52 is configured to couple to an actuation mechanism of the surgical instrument 50 and/or the camera 51 and is configured to move (e.g., rotate) and actuate the surgical instrument 50 and/or the camera 51. IDU 52 transfers actuation forces from its actuators to the surgical instrument 50 to actuate components an end effector 49 of the surgical instrument 50. The holder 46 includes a sliding mechanism 46a, which is configured to move the IDU 52 along the second longitudinal axis defined by the holder 46. The holder 46 also includes a joint 46b, which rotates the holder 46 relative to the link 42c. During procedures, the surgical instrument 50 or camera 51 may be inserted through an access port 55 (FIG. 3) held by the holder 46. The holder 46 also includes a port latch 46c for securing the access port 55 to the holder 46 (FIG. 2). In aspect, the camera 51 and access port 55 may be components of a laparoscopic system 100 (FIG. 6).

The robotic arm 40 also includes a plurality of manual override buttons 53 (FIG. 1) disposed on the IDU 52 and the setup arm 61, which may be used in a manual mode. The user may press one or more of the buttons 53 to move the component associated with the button 53.

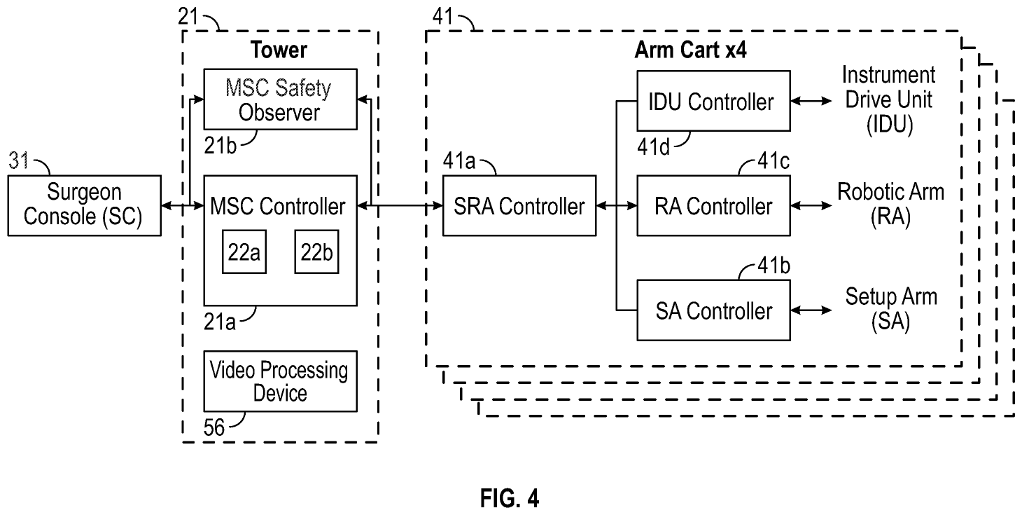

With reference to FIG. 4, each of the computers 21, 31, 41 of the surgical robotic system 10 may include a plurality of controllers, which may be embodied in hardware and/or software. The computer 21 of the control tower 20 includes a controller 21a and safety observer 21b. The controller 21a receives data from the computer 31 of the surgeon console 30 about the current position and/or orientation of the hand controllers 38a and 38b and the state of the foot pedals 36 and other buttons. The controller 21a processes these input positions to determine desired drive commands for each joint of the robotic arm 40 and/or the IDU 52 and communicates these to the computer 41 of the robotic arm 40. The controller 21a also receives the actual joint angles measured by encoders of the actuators 48a and 48b and uses this information to determine force feedback commands that are transmitted back to the computer 31 of the surgeon console 30 to provide haptic feedback through the hand controllers 38a and 38b. The safety observer 21b performs validity checks on the data going into and out of the controller 21a and notifies a system fault handler if errors in the data transmission are detected to place the computer 21 and/or the surgical robotic system 10 into a safe state.

The controller 21a is coupled to a storage 22a, which may be non-transitory computer-readable medium configured to store any suitable computer data, such as software instructions executable by the controller 21a. The controller 21a also includes transitory memory 22b for loading instructions and other computer readable data during execution of the instructions. In embodiments, other controllers of the surgical robotic system 10 include similar configurations.

The computer 41 includes a plurality of controllers, namely, a main cart controller 41a, a setup arm controller 41b, a robotic arm controller 41c, and an instrument drive unit (IDU) controller 41d. The main cart controller 41a receives and processes joint commands from the controller 21a of the computer 21 and communicates them to the setup arm controller 41b, the robotic arm controller 41c, and the IDU controller 41d. The main cart controller 41a also manages instrument exchanges and the overall state of the mobile cart 60, the robotic arm 40, and the IDU 52. The main cart controller 41a also communicates actual joint angles back to the controller 21a.

Each of joints 63a and 63b and the rotatable base 64 of the setup arm 61 are passive joints (i.e., no actuators are present therein) allowing for manual adjustment thereof by a user. The joints 63a and 63b and the rotatable base 64 include brakes that are disengaged by the user to configure the setup arm 61. The setup arm controller 41b monitors slippage of each of joints 63a and 63b and the rotatable base 64 of the setup arm 61, when brakes are engaged or can be freely moved by the operator when brakes are disengaged, but do not impact controls of other joints. The robotic arm controller 41c controls each joint 44a and 44b of the robotic arm 40 and calculates desired motor torques required for gravity compensation, friction compensation, and closed loop position control of the robotic arm 40. The robotic arm controller 41c calculates a movement command based on the calculated torque. The calculated motor commands are then communicated to one or more of the actuators 48a and 48b in the robotic arm 40. The actual joint positions are then transmitted by the actuators 48a and 48b back to the robotic arm controller 41c.

The IDU controller 41d receives desired joint angles for the surgical instrument 50, such as wrist and jaw angles, and computes desired currents for the motors in the IDU 52. The IDU controller 41d calculates actual angles based on the motor positions and transmits the actual angles back to the main cart controller 41a.

The robotic arm 40 is controlled in response to a pose of the hand controller controlling the robotic arm 40, e.g., the hand controller 38a, which is transformed into a desired pose of the robotic arm 40 through a hand eye transform function executed by the controller 21a. The hand eye function, as well as other functions described herein, is/are embodied in software executable by the controller 21a or any other suitable controller described herein. The pose of one of the hand controllers 38a may be embodied as a coordinate position and roll-pitch-yaw (RPY) orientation relative to a coordinate reference frame, which is fixed to the surgeon console 30. The desired pose of the surgical instrument 50 is relative to a fixed frame on the robotic arm 40. The pose of the hand controller 38a is then scaled by a scaling function executed by the controller 21a. In embodiments, the coordinate position may be scaled down and the orientation may be scaled up by the scaling function. In addition, the controller 21a may also execute a clutching function, which disengages the hand controller 38a from the robotic arm 40. In particular, the controller 21a stops transmitting movement commands from the hand controller 38a to the robotic arm 40 if certain movement limits or other thresholds are exceeded and in essence acts like a virtual clutch mechanism, e.g., limits mechanical input from effecting mechanical output.

The desired pose of the robotic arm 40 is based on the pose of the hand controller 38a and is then passed by an inverse kinematics function executed by the controller 21a. The inverse kinematics function calculates angles for the joints 44a, 44b, 44c of the robotic arm 40 that achieve the scaled and adjusted pose input by the hand controller 38a. The calculated angles are then passed to the robotic arm controller 41c, which includes a joint axis controller having a proportional-derivative (PD) controller, the friction estimator module, the gravity compensator module, and a two-sided saturation block, which is configured to limit the commanded torque of the motors of the joints 44a, 44b, 44c.

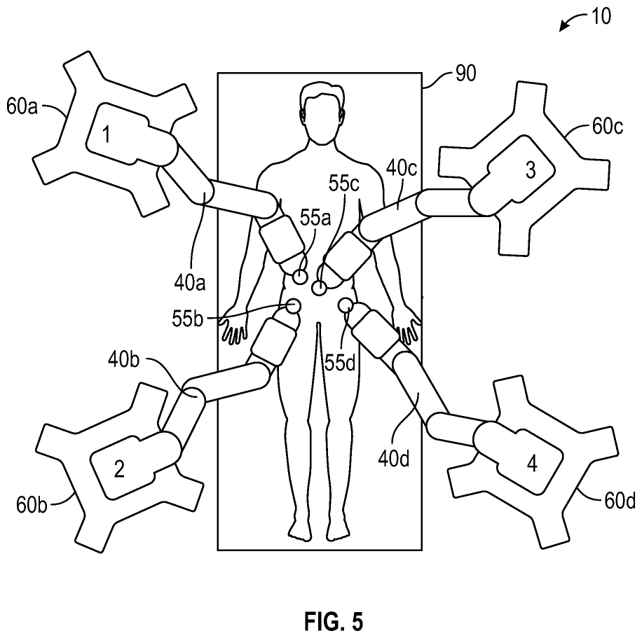

With reference to FIG. 5, the surgical robotic system 10 is setup around a surgical table 90. The surgical robotic system 10 includes mobile carts 60a-d, which may be numbered “1” through “4.” During setup, each of the carts 60a-d are positioned around the surgical table 90. Position and orientation of the carts 60a-d depends on a plurality of factors, such as placement of a plurality of access ports 55a-d, which in turn, depends on the surgery being performed. Once the port placement is determined, the access ports 55a-d are inserted into the patient, and carts 60a-d are positioned to insert surgical instruments 50 and the laparoscopic camera 51 into corresponding ports 55a-d.

During use, each of the robotic arms 40a-d is attached to one of the access ports 55a-d that is inserted into the patient by attaching the latch 46c (FIG. 2) to the access port 55 (FIG. 3). The IDU 52 is attached to the holder 46, followed by the SIM 43 being attached to a distal portion of the IDU 52. Thereafter, the surgical instrument 50 is attached to the SIM 43. The surgical instrument 50 is then inserted through the access port 55 by moving the IDU 52 along the holder 46. The SIM 43 includes a plurality of drive shafts configured to transmit rotation of individual motors of the IDU 52 to the surgical instrument 50 thereby actuating the surgical instrument 50. In addition, the SIM 43 provides a sterile barrier between the surgical instrument 50 and the other components of robotic arm 40, including the IDU 52. The SIM 43 is also configured to secure a sterile drape (not shown) to the IDU 52.

In accordance with aspects of the disclosure, and with reference to FIGS. 6 and 7, a laparoscopic system 100 (FIG. 6) and a laparoscope 400 (FIG. 7) are provided and described in detail below. The laparoscopic system 100 and laparoscope 400 may be standalone, manually operated components, or may be integrated into surgical robotic system 10. For example, the access port 200 and the laparoscope 300 of laparoscopic system 100 may correspond to the access port 55 (FIG. 3) and the camera 51 (FIG. 1), respectively, described above with respect to surgical robotic system 10. In other aspects, the access port 200 and the laparoscope 300 of laparoscopic system 100 are handheld and manually operable components configured to be manually manipulated by a clinician without the use of some, or any, of the components of surgical robotic system 10. Likewise, the laparoscope 400 (FIG. 7) may correspond to the camera 51 (FIG. 1) of surgical robotic system 10 or may be a handheld and manually operable component configured to be manually operated by a clinician without the use of some, or any, of the components of surgical robotic system 10.

With specific reference to FIG. 6, laparoscopic system 100 includes an access port 200 and a laparoscope 300 configured to be inserted through the access port 200 to gain access to a surgical site. The access port 200 includes a proximal portion 200a, a distal portion 200b, and an elongated body 220 extending between the proximal portion 200a and the distal portion 200b. The distal portion 200b may have a tapered or sharpened distal tip to assist in inserting or puncturing the access port 200 through tissue. In aspects, the access port 200 may include one or more valve systems 230 for connecting the access port 200 to one or more sources of suction and/or insufflation. The proximal portion 200a of the access port 200 defines a proximal opening 260 through which a portion of the laparoscope 300 is configured to be inserted.

A magnet 240, which generates a magnetic field, is operably coupled to a portion of the access port 200. In an aspect, the magnet 240 is positioned in the proximal portion 200a of the access port 200, for example, within a proximal housing 210 of the access port 200. The magnet 240 may be a ring magnet defining an opening 240a which is aligned with the proximal opening 260 of the access port 200. In such a configuration, the portion of the laparoscope 300 that passes through the proximal opening 260 of the access port 200 when the laparoscope 300 is inserted through the access port 200 also passes through the opening 240a of the magnet 240 thereby exposing portions of the laparoscope 300 to the magnetic field generated by the magnet 240.

The laparoscope 300 has a proximal portion 300a, a distal portion 300b, and an elongated body 320 extending between the proximal portion 300a and the distal portion 300b. The elongated body 320 of the laparoscope 300 has an outer diameter smaller than the inner diameter of the elongated body 220 of the access port 200 so that the elongated body 320 can pass through the elongated body 220 of the access port 200.

The laparoscope 300 includes a camera module 370 which is configured to capture video of the surgical site and transmit the video, via a wired connection or wirelessly, for storage and/or viewing. For example, the camera module 370 may be coupled to the video processing device 56 (FIG. 4), which may be disposed within the control tower 20 or surgeon console 30. The video processing device 56 may be any computing device configured to receive the video feed from the camera module 370 of the laparoscope 300 and output the processed video stream for display on a display and/or store the video stream within a memory. In aspects, the camera module 370 of laparoscope 300 includes an internal video processing device which performs some or all of the same functions as video processing device 56.

The laparoscope 300 includes a magnetic sensor 340 operably coupled to a portion of the proximal portion 300a, distal portion 300b, or a point along the length of the elongated body 320. In aspects, the magnetic sensor 340 is disposed at a point proximate the distal portion 300b of the laparoscope 300. In other aspects, the magnetic sensor 340 is disposed at a point proximate the proximal portion 300a of the laparoscope 300. The magnetic sensor 340 may be a reed switch, hall effect sensor, or any other sensor capable of transmitting a signal upon being exposed to a magnetic field. Conductors 360 extend from the magnetic sensor 340 to conductor contacts 360p located at the proximal portion 300a of the laparoscope 300. The conductor contacts 360p are aligned with conductor pins 370p of the camera module 370 such that, when the camera module 370 is connected to the proximal portion 300a of the laparoscope 300, the conductor contacts 360p are in conductive contact with the conductor pins 370p. Either or both of conductor contacts 360p or conductor pins 370p may be spring-loaded.

Upon insertion of the laparoscope 300 through the access port 200, the magnetic sensor 340 is exposed to the magnetic field generated by the magnet 240 of the access port 200. The magnetic field activates the magnetic sensor 340 and current flow generates a signal which is transmitted from the magnetic sensor 340, through the conductors 360, and to the camera module 370. The signal transmitted to the camera module 370 causes the camera module 370 to switch between a first mode of operation and a second mode of operation.

In a first mode of operation, the camera module 370 functions in a manner that reduces power consumption, reduces bandwidth consumption, and/or reduces memory consumption. The resulting effect of operating in the first mode is less consumption of system and components resources, faster processing, extended life of the components, and increased privacy of the patient and individuals in the surgical setting. For example, when operating in the first mode of operation, the camera module 370 may be prevented from capturing video, may capture video at a relatively low resolution, may be prevented from storing captured video within the camera module 370, or may be prevented from transmitting the video feed to an external component for storage or display. When operating in the first mode of operation, the resolution of the video feed can be reduced to a level that would result in the objects and individuals in the video feed being unrecognizable, thereby protecting the privacy of any individuals captured in the video feed. In addition, when the resolution of the video feed is reduced, the size of the data of the video feed is also reduced. Therefore, any lower resolution video, even if recorded, would consume less memory within a storage device than that of a full resolution video feed (e.g., the resolution of the video feed while in the second mode of operation). Likewise, the transmission and processing of the lower resolution video feed consumes less bandwidth than that of a higher resolution video feed (e.g., the resolution of the video feed while in the second mode of operation).

In the second mode of operation, the camera module 370 may initiate the capture, recording, or transmission of video or, in some aspects, may additionally or alternatively increase the resolution of the video captured to a normal or high resolution suitable for a surgical setting. For example, when the camera module 370 is prevented from capturing video in the first mode of operation, then in the second mode of operation, the camera module 370 initiates the capture of the video. As an additional example, when the camera module 370 captures video at a relatively low resolution in the first mode of operation, then in the second mode of operation, the camera module 370 increases the resolution of the captured video to a higher resolution. As yet another example, when the camera module 370 is prevented from recording or storing the captured video in the first mode of operation, then in the second mode of operation, the camera module 370 initiates the recording or storage of the captured video.

In aspects, the camera module 370 remains in the first mode of operation until the laparoscope 300 is inserted into the surgical site through the access port 200, that is, until the camera module 370 is switched to the second mode of operation. In particular, the camera module 370 may be configured to initially operate in the first mode of operation until the magnetic sensor 340 is positioned within the magnetic field generated by the magnet 240 of the access port 200 and causes the camera module 370 to switch from the first mode of operation to the second mode of operation. In an aspect, in configurations where the camera module 370 is not capturing, transmitting, or storing the video feed in the first mode of operation (e.g., the camera module is “off”), switching the camera module 370 to the second mode of operation simply initiates the capture, transmission, and/or storage of the video feed by the camera module 370 (e.g., switches the camera module 370 “on”). In another aspect, in configurations where the camera module 370 is capturing, transmitting, or storing the video feed in a first (lower) resolution in the first mode of operation, switching the camera module 370 to the second mode of operation causes the camera module 370 to initiate the capture, transmission, and/or storage of the video feed in a higher resolution than the resolution of the first mode of operation.

In aspects, exposure of the magnetic sensor 340 to the magnetic field generated by the magnet 240 for a period of time exceeding a predetermined threshold initiates the switch between the first mode of operation and the second mode of operation and the camera module 370 remains in the second mode of operation even while the magnetic sensor 340 is not within the magnetic field. In such an aspect, the camera module 370 may switch back to the first mode of operation from the second mode of operation once the magnetic sensor 340 is again exposed to the magnetic field for a period of time exceeding a predetermined threshold. The predetermined threshold for switching the camera module 370 from the first mode of operation to the second mode of operation may be the same as or different from the predetermined threshold for switching the camera module 370 from the second mode of operation to the first mode of operation. Alternatively, or in combination with the aspects described above, the camera module 370 will remain in the second mode of operation only while the magnetic sensor 340 is exposed to the magnetic field generated by the magnet 240 and once the magnetic sensor 340 is removed from the magnetic field, the camera module 370 reverts back to the first mode of operation.

The embodiment of laparoscopic system 100, described above in connection with FIG. 6, is invariant to the lighting conditions and does not rely on an analysis of the images captured to detect when the laparoscope 300 is positioned in the surgical site.

FIG. 7 illustrates a laparoscope 400 which includes a switching system that does not require an external component such as an access port (e.g., access port 200 of FIG. 6) to switch between a first mode of operation and a second mode of operation. However, in aspects, laparoscope 400 may be a part of a laparoscopic system that includes an access port through which the laparoscope may be inserted, like that of laparoscopic system 100 (FIG. 6).

The laparoscope 400 has a proximal portion 400a, a distal portion 400b, and an elongated body 420 extending between the proximal portion 400a and the distal portion 400b. The laparoscope 400 includes a camera module 470 which is configured to capture video of the surgical site and transmit the video, via a wired connection or wirelessly, for storage and/or viewing. For example, the camera module 470 may be coupled to the video processing device 56 (FIG. 4), which may be disposed within the control tower 20 or surgeon console 30. The video processing device 56 may be any computing device configured to receive the video feed from the camera module 470 of the laparoscope 400 and output the processed video stream for display on a display and/or store the video stream within a memory. In aspects, the camera module 470 of laparoscope 400 includes an internal video processing device which performs some or all of the same functions as video processing device 56.

The laparoscope 400 includes a light sensor 480 operably coupled to a portion of the proximal portion 400a, distal portion 400b, or a point along the length of the elongated body 420. In aspects, the light sensor 480 is disposed at a point proximate the distal portion 400b of the laparoscope 400. In other aspects, the light sensor 480 is disposed at a point proximate the proximal portion 400a of the laparoscope 400. The light sensor 480 may be a photo resistor, light dependent resistor, or any other such sensor capable of transmitting or generating a signal based on its exposure to light. In an aspect, the laparoscope 400 includes an opening 440 located at the distal portion 400b of the laparoscope 400, though the opening 440 may be located at any position along the length of the elongated body 420 that is configured to be positioned within the surgical site. The opening 440 may be located in consideration of any illuminating elements included in the laparoscope 400 that have the potential to affect the amount of light surrounding and entering the opening 440. For example, in aspects where the laparoscope 400 includes an illuminating element (e.g., at the distal portion 400b) to illuminate the surgical site, it is appreciated that the projection of light is directionally forward-facing and any illumination before the projecting end is relatively lower, and therefore the opening 440 may be located before (e.g., proximal) the projecting end. An optical fiber 460 extends from the opening 440 and to the light sensor 480 such that light may pass through the optical fiber 460 from the opening 440 and to the light sensor 480.

During the course of a procedure, the light content is relatively higher outside of the surgical site than within the surgical site. Thus, when the level of light detected by the light sensor 480 transitions from a first (higher) level to a second (lower) level, such as when the laparoscope 400 is inserted through an access port or is otherwise positioned within a surgical site, the camera module 470 is caused to switch from a first mode of operation to a second mode of operation.

In a first mode of operation, the camera module 470 functions in a manner that reduces power consumption, reduces bandwidth consumption, and/or reduces memory consumption. The resulting effect of operating in the first mode is less consumption of system and components resources, faster processing, extended life of the components, and increased privacy of the patient and individuals in the surgical setting. For example, when operating in the first mode of operation, the camera module 470 may be prevented from capturing video, may capture video at a relatively low resolution, may be prevented from storing captured video within the camera module 470, or may be prevented from transmitting the video feed to an external component for storage or display. When operating in the first mode of operation, the resolution of the video feed can be reduced to a level that would result in the objects and individuals in the video feed being unrecognizable, thereby protecting the privacy of any individuals captured in the video feed. In addition, when the resolution of the video feed is reduced, the size of the data of the video feed is also reduced. Therefore, any lower resolution video, even if recorded, would consume less memory within a storage device than that of a full resolution video feed (e.g., the resolution of the video feed while in the second mode of operation). Likewise, the transmission and processing of the lower resolution video feed consumes less bandwidth than that of a higher resolution video feed (e.g., the resolution of the video feed while in the second mode of operation).

In the second mode of operation, the camera module 470 may initiate the capture, recording, or transmission of video or, in some aspects, may additionally or alternatively increase the resolution of the video captured to a normal or high resolution suitable for a surgical setting. For example, when the camera module 470 is prevented from capturing video in the first mode of operation, then in the second mode of operation, the camera module 470 initiates the capture of the video. As an additional example, when the camera module 470 captures video at a relatively low resolution in the first mode of operation, then in the second mode of operation, the camera module 470 increases the resolution of the captured video to a higher resolution. As yet another example, when the camera module 470 is prevented from recording or storing the captured video in the first mode of operation, then in the second mode of operation, the camera module 470 initiates the recording or storage of the captured video.

In aspects, the camera module 470 remains in the first mode of operation until the laparoscope 400 is inserted into the surgical site, that is, until the camera module 470 is switched to the second mode of operation. In particular, the camera module 470 may be configured to initially operate in the first mode of operation until the light sensor 480 senses a reduction in the level of light that exceeds a predetermined threshold and causes the camera module 470 to switch from the first mode of operation to the second mode of operation. In an aspect, in configurations where the camera module 470 is not capturing, transmitting, or storing the video feed in the first mode of operation (e.g., the camera module 470 is “off”), switching the camera module 470 to the second mode of operation simply initiates the capture, transmission, and/or storage of the video feed by the camera module 470 (e.g., switches the camera module 470 “on”). In another aspect, in configurations where the camera module 470 is capturing, transmitting, or storing the video feed in a first (lower) resolution in the first mode of operation, switching the camera module 470 to the second mode of operation causes the camera module 470 to initiate the capture, transmission, and/or storage of the video feed in a higher resolution than the resolution of the first mode of operation.

In aspects, the camera module 470 may switch back to the first mode of operation from the second mode of operation once the light sensor 480 is again exposed to the same level of light (within a predetermined margin) to which it was exposed while the camera module 470 was in the first mode of operation (e.g., before the camera module 470 was switched to the second mode of operation). The predetermined threshold for switching the camera module 470 from the first mode of operation to the second mode of operation may be the same as or different from the predetermined threshold for switching the camera module 470 from the second mode of operation to the first mode of operation.

It will be understood that various modifications may be made to the embodiments disclosed herein. Therefore, the above description should not be construed as limiting, but merely as exemplifications of various embodiments. Those skilled in the art will envision other modifications within the scope and spirit of the claims appended thereto.

What is claimed is:

1. A laparoscopic system comprising:
an access port including a magnet operably coupled to a portion of the access port; and
a laparoscope configured to be inserted through the access port to access a surgical site, the laparoscope including:
a camera module configured to capture video of the surgical site and switchable between a first mode and a second mode; and
a magnetic sensor operably coupled to the camera module and configured to cause the camera module to switch from operating in the first mode to operating in the second mode when the magnetic sensor is positioned in proximity to the magnet of the access port.

2. The laparoscopic system of claim 1, wherein a proximal portion of the access port defines a proximal opening configured to receive a distal portion of the laparoscope therethrough.

3. The laparoscopic system of claim 2, wherein the magnet is a ring magnet defining an opening aligned with the proximal opening of the access port.

4. The laparoscopic system of claim 1, wherein the magnetic sensor includes at least one of a reed switch or a hall effect sensor.

5. The laparoscopic system of claim 1, wherein the magnetic sensor is operably coupled to a distal portion of the laparoscope and the magnet is disposed at a proximal portion of the access port, such that the magnetic sensor is configured to cause the camera module to switch from operating in the first mode to operating in the second mode upon insertion of the distal portion of the laparoscope through the proximal portion of the access port.

6. The laparoscopic system of claim 1, wherein the camera module is configured to capture video when in the second mode and not capture video when in the first mode.

7. The laparoscopic system of claim 1, wherein the camera module is configured to transmit captured video when in the second mode and not transmit captured video when in the first mode.

8. The laparoscopic system of claim 1, wherein the camera module is configured to transmit video in a first resolution when in the second mode and transmit video in a second resolution when in the first mode, the first resolution being higher than the second resolution.

9. A laparoscope comprising:
a proximal portion;
a distal portion including an opening;
an elongated body extending between the proximal portion and the distal portion;
a camera module including a light sensor, the camera module configured to capture video of a surgical site and switchable between a first mode and a second mode; and
an optical fiber extending from the camera module, through the elongated body, and to the opening of the distal portion and configured to transmit light passing through the opening to the light sensor, wherein the light sensor switches the camera module between operating in the first mode and operating in the second mode based on an amount light to which the light sensor is exposed.

10. The laparoscope of claim 9, wherein the light sensor is a light dependent resistor.

11. The laparoscope of claim 9, wherein the camera module is configured to capture video when in the second mode and not capture video when in the first mode.

12. The laparoscope of claim 9, wherein the camera module is configured to transmit captured video when in the second mode and not transmit captured video when in the first mode.

13. The laparoscope of claim 9, wherein the camera module is configured to transmit video in a first resolution when in the second mode and transmit video in a second resolution when in the first mode, the first resolution being higher than the second resolution.

14. A laparoscope comprising:
a camera module configured to capture video of a surgical site and switchable between a first mode and a second mode; and
at least one of:
a magnetic sensor operably coupled to the camera module and configured to cause the camera module to switch from operating in the first mode to operating in the second mode when the magnetic sensor is positioned in proximity to a magnet of an access port; or
a light sensor operably coupled to the camera module and an optical fiber operably coupled to the light sensor, wherein the light sensor is configured to cause the camera module to switch between operating in the first mode and operating in the second mode based on an amount light to which the light sensor is exposed;
wherein the camera module is configured to at least one of:
capture video when in the second mode and not capture video when in the first mode;
transmit captured video when in the second mode and not transmit captured video when in the first mode;
transmit video in a first resolution when in the second mode and transmit video in a second resolution when in the first mode, the first resolution being higher than the second resolution; or
stop a recording of the captured video when switched from the second mode of operation to the first mode of operation.

15. The laparoscope of claim 14, wherein a distal portion of the laparoscope is configured to extend through a ring magnet of an access port to expose the magnetic sensor to a magnetic field of the ring magnet.

16. The laparoscope of claim 14, wherein the magnetic sensor includes at least one of a reed switch or a hall effect sensor.

17. The laparoscope of claim 14, wherein the light sensor is a light dependent resistor.

18. The laparoscope of claim 14, wherein a distal portion of the laparoscope includes an opening and a distal end of the optical fiber is disposed at the opening.

19. The laparoscope of claim 14, wherein the camera module operates in the first mode until it is switched to the second mode.
